(19) United States
(12) Patent Application Publication
(10) Pub. No.: US 2009/0069706 A1
(43) Pub. Date: Mar. 12, 2009

(54) BRAIN PROBE ADAPTED TO BE INTRODUCED THROUGH A CANULA

(76) Inventors: Jerome Boogaard, Forest Grove, OR (US); Robert Lucas, Prineville, OR (US); Mike DeGiacomo, Portland, OR (US); Sergey Varivoda, Oregon City, OR (US)

Correspondence Address: ST. JUDE MEDICAL NEUROMODULATION, 6901 PRESTON ROAD, PLANO, TX, 75024, US

(21) Appl. No.: 12/268,875

(22) Filed: Nov. 11, 2008

Related U.S. Application Data
(63) Continuation of application No. 11/580,740, filed on Oct. 12, 2006, now abandoned.
Application No. 11/580,740 is a continuation-in-part of application No. 10/760,856, filed on Jan. 20, 2004, now Pat. No. 7,190,989.
Application No. 10/760,856 is a continuation-in-part of application No. 10/429,652, filed on May 5, 2003, now Pat. No. 6,892,438.
Application No. 10/429,652 is a division of application No. 09/886,322, filed on Jun. 21, 2001, now Pat. No. 6,560,472.

(51) Int. Cl. A61B 5/0476

(52) U.S. Cl. 600/544

(57) ABSTRACT

A bio-probe having a base and a tip and also including a set of at least four conductors extending longitudinally along the bio-probe, the conductors being coated with dielectric material. Also, at least four of the conductors define a spot where the dielectric material has been removed, thereby defining an electrical contact site. In addition, the bio-probe is less than 2.5 mm thick in its greatest transverse dimension along a longitudinal portion extending from the tip to a point 6 cm proximal of the tip.

### RELATED APPLICATION

This application is a continuation-in-part of application Ser. No. 10/760,856, filed Jan. 20, 2004, which is a continuation in part of application Ser. No. 10/429,652 filed May 5, 2003, now U.S. Pat. No. 6,892,438, issued May 17, 2005, which is a divisional of Ser. No. 09/886,322, filed Jun. 21, 2001, now U.S. Pat. No. 6,560,472, issued May 6, 2003.

### STATEMENT OF GOVERNMENTAL SUPPORT

This invention was made with government support under grant No. 1R43 MH59502-01 awarded by the Small Business Research Program of the Department of Health and Human Services of the Public Health Service. The government has certain rights in the invention.

### BACKGROUND OF THE INVENTION

The construction of a brain probe assembly to be employed in brain research is quite challenging from both a structural and an electrical standpoint.

Structurally, probes must not fray or in any way come apart when pushed through the dura, a tough membrane covering the brain, and other brain tissue. Probes should have enough strength and rigidity to broach the dura without the need for assistance by, for example, a guide tube or an initial incision.

Moreover, probes must not break, running the risk of leaving a fragment in the brain. Also, they must not cause undue damage to tissue at the sensing site. Inevitably, the tissue separating the sensing site from the brain exterior will suffer some damage as a probe is pushed to its destination. A small cross-section probe, however, may cause less damage as it is pushed to its destination. It is best to avoid having a sharp tip or any sharp edges, however, as this could cause blood vessels to be severed during the insertion process.

Electrically, one should note that the electric field signals in the brain, which the probe is designed to detect, are typically of the order of 100 to 500 μvolts. The low amplitude of these signals makes it necessary to amplify them as physically close as possible to their source. In fact, the signals involved are so minute that variations in circuit geometry could well affect significantly the detection processing of the signals. It is also highly desirable to minimize cross-talk between any two signals.

Additionally, it is generally advantageous for a brain probe to become flexible after being inserted so that the motion of the brain within the brain pan is not resisted by the probe. In the worst case this could cause tissue tearing. To insert a brain probe, however, it is better for the probe to be in a rigid state. Given the tight geometries allowable for brain probe design, these requirements are difficult to meet simultaneously.

Further, a tube-like device known as a canula is generally used for inserting devices into the brain. Being able to fit through a canula, to a desired depth, is an important and desirable attribute of a brain probe.

### SUMMARY OF THE INVENTION

In a first, separate aspect, the present invention may take the form of a brain probe that includes a core having a distal end and a proximal end and a dielectric coating, over the core. In addition, a set of traces are located on the dielectric coating and a dielectric layer that is deposited over the traces defines an aperture for each trace, wherein the trace is exposed, thereby constituting an electrical contact point. Also, a set of insulated wires is each electrically connected to a trace and a conductive tube is affixed to the proximal end of the core, covering the wires.

In a second separate aspect, the present invention is a bio-probe having a base and a tip and also including a set of at least four conductors extending longitudinally along the bio-probe, the conductors being coated with dielectric material. Also, at least four of the conductors define a spot where the dielectric material has been removed, thereby defining an electrical contact site. Also, the bio-probe is less than 2 mm thick in its greatest transverse dimension along a longitudinal portion extending from the tip to a point 6 cm proximal of the tip.

In a third separate aspect, the present invention is a brain probe assembly for use accessing a target brain type to a target depth. The assembly includes a canula and a brain probe having a distal portion sized to fit through the canula and to extend into the target brain type to the target depth. Also, the brain probe includes 4 mutually electrically isolated electrical contact points.

The foregoing and other objectives, features and advantages of the invention will be more readily understood upon consideration of the following detailed description of the preferred embodiment(s), taken in conjunction with the accompanying drawings.

### BRIEF DESCRIPTION OF THE DRAWINGS

FIG. 1 is an exploded perspective view of bio-probe assembly according to the present invention.

FIG. 2 is a front view of the circuit card assembly of the bio-probe assembly of claim 1.

FIG. 3 is an expanded perspective view of the tip of the bio-probe assembly of FIG. 1.

FIG. 4 is a greatly expanded cross-sectional view of the tip of the bio-probe assembly of FIG. 1.

FIG. 5 is a side view of an alternative embodiment of a bio-probe, according to the present invention.

FIG. 6 is a cross-sectional view of the bio-probe of FIG. 5, taken along line 6-6 of FIG. 5.

FIG. 7 is a side view of an alternative embodiment of a brain probe, according to the present invention.

FIG. 8 is a side view of the brain probe of FIG. 7, inserted into a brain and intersecting a brain organ.

FIG. 9 is a sectional view of the brain probe of FIG. 7, taken along view line 9-9.

FIG. 10 is a side view of a wire holding ring, an element of the brain probe of FIG. 7.

FIG. 11 is an expanded side cut-away of a portion of the brain probe of FIG. 7.

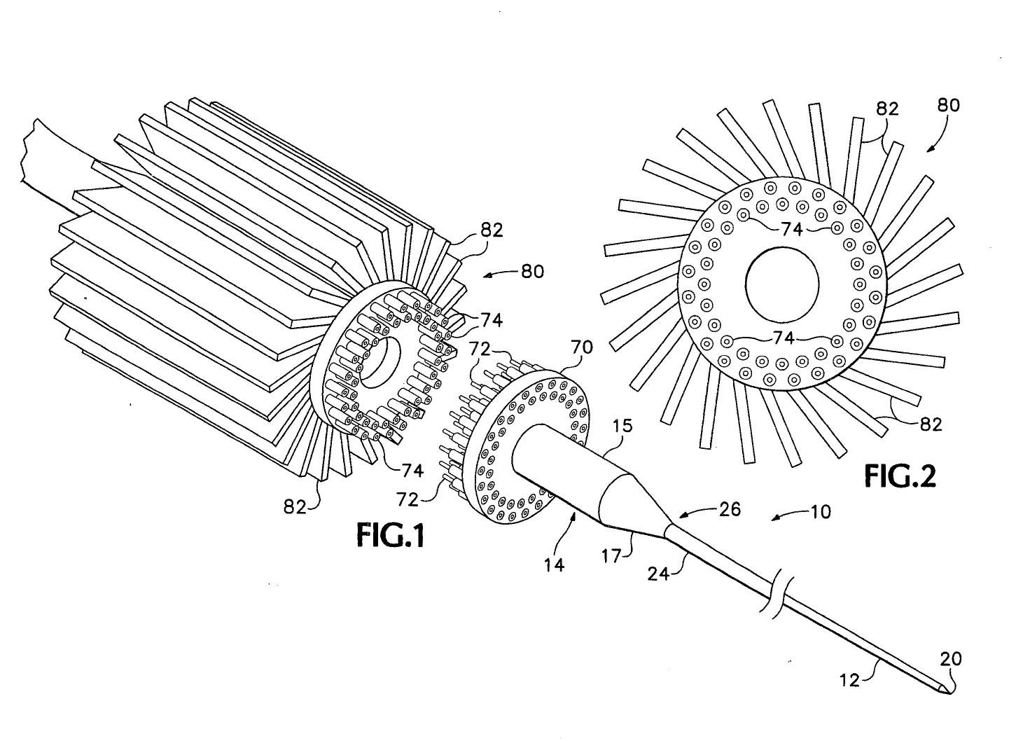

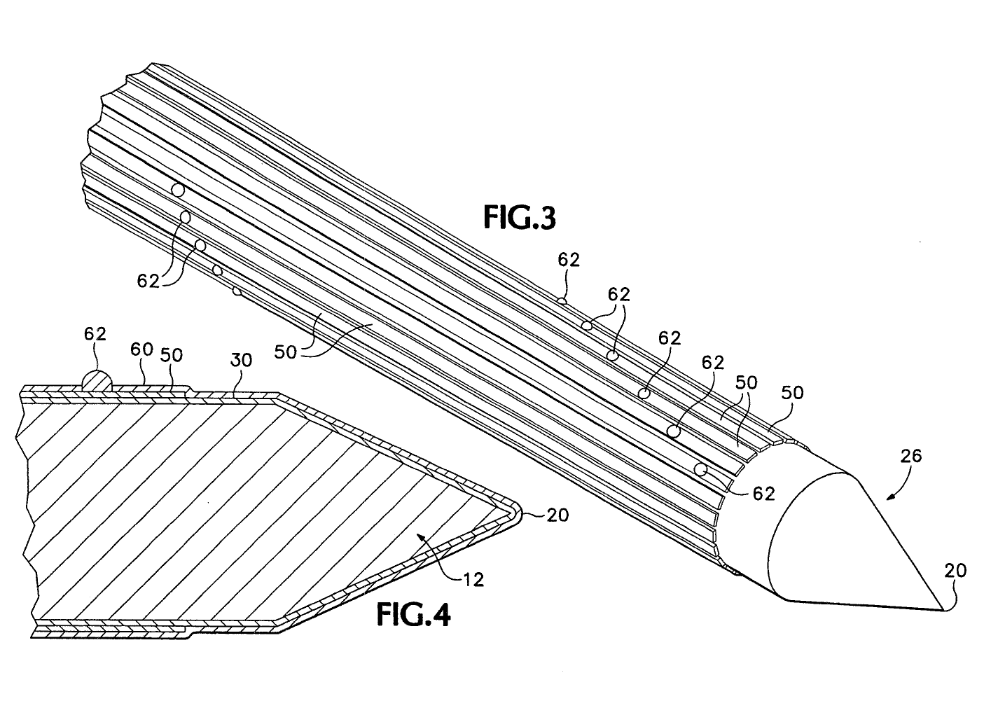

### DETAILED DESCRIPTION OF THE PREFERRED EMBODIMENT

A preferred embodiment of a brain probe or bio-probe assembly 10, according to the present invention is composed of a probe core 12 and a handle core 14. The probe core 12 is made of tungsten, chosen for its material stiffness and tensile strength. Probe core 12 is preferably straight. To achieve this end, a straightening machine that pulls on core 12, thereby creating tensile stress and annealing core 12 may be used. During further operations, a vacuum chuck may be used to hold core 12 in place. A tip or distal end 20 of probe core 12 has a diameter of 200 microns (8.0 mils) and a base or proximal end 24 of core 12 has a diameter of 600 microns (24 mils). In addition, core 12 is 89 mm (3.5″) long. The tip 20 is preferably formed by way of centerless grinding. Probe core 12 should be electro polished so that the deposition of materials onto it (see below) can be accomplished efficiently and so that the finished assembly 10 can pass through brain tissue as smoothly as possible. Alternatively, probe core 12 can be left in a comparatively rough state and coated with a coat of epoxy that is thick enough to minimize capacitance between core 12 and the traces 50 (discussed below). The comparatively rough state of the probe core actually helps to effect the binding of the epoxy to the probe core. One type of epoxy that can be used is the epoxy 377 discussed further below.

For ease of assembly, and so that operating personnel may more easily handle assembly 10, the handle core 14 is expanded in cross-section relative to probe core 12. Although the handle core 14 is preferably a unitary piece of medical grade 304 stainless steel, it may be conceptually divided into a cylinder 15, having a diameter of 4.826 mm (0.19″), and a frustum 17. The frustum 17 tapers inwardly at 15° angle from the sides of cylinder 15. A 600 μm (24 mil) aperture (not shown) at the narrow end of frustum 17 permits introduction of the base of probe core 12, after which probe core 12 is joined to handle core 14, by way of an epoxy, to form joint core 26. The epoxy used must be conductive, so that the probe core 12 is grounded to the base core 14, and preferably heat resistant, so that it withstands the sterilization process that the probe 10 generally should undergo in use. It must also be able to withstand the different degrees of expansion that stainless steel and tungsten undergo during the sterilization process. An epoxy that is available from Epoxy Technology, Inc. of Billerica, Mass., under the designation E3084 appears to meet these requirements. In an alternative preferred embodiment, the probe core 12 is laser-welded to the base core 14.

After joint core 26 is produced, it is dip coated with a dielectric epoxy, which has been premixed with a surfactant to promote an even coating, to form an insulating coat 30. The desirable characteristics for an epoxy to be used are biocompatibility, heat tolerance to withstand the sterilization process, low viscosity to produce a thin film, a heat accelerated cure, and a high bulk resistivity, and a low dielectric coefficient to avoid electrical losses and withstand electrostatic charges. One epoxy that appears to meet these requirements is the epoxy #377 noted earlier. A suitable surfactant is available as FC-430 from 3M of St. Paul, Minn. Alternatively, acrylated epoxy could be used. For coat 30, this material could have the composition, noted in Table I, below, in parts per hundred resin (PHR):

TABLE I

| Substance | Proportion | Source, Contact Information |
|---|---|---|
| Photomer 3015 | 100 PHR | Cognis Corp., |
| | | http://www.na.cognis.com/northamerica/nacognis.html |
| TMPEOTA, | 50 PHR | Sartomer Company, Inc., http://www.sartomer.com |
| (Trimethylolpropane | | |
| triacrylate SR-351) | | |
| R-812S (fumed | 10 PHR | Degussa Corp., http://www.degussa.com/en/home.html |
| silica) | | |
| MIBK (Methyl | 20 PHR | Aldrich Corp., |
| Isobutyl Keytone) | | http://www.sigmaaldrich.com/Brands/Aldrich.html |
| Darocure 1173 | 2.6 PHR | EM Chemicals Corp., |
| (Photoinitiator) | | http://www.emdchemicals.com/corporate/emd_corporate.asp |

In an additional preferred embodiment quartz crystal, glass or a similar dielectric material is vacuum deposited to form coat 30. In this preferred embodiment, in order to gain adherence, however, a 200 Å coat of chrome (not shown) is first applied, also through vacuum deposition on core 26 to promote the adhesion of coat 30. The thickness of coat 30 is chosen to minimize the capacitance between core 26 and the conductive traces 50 (see below) deposited over it.

On top of coat 30, a 0.5 μm thick plate of conductive material (not shown as such but later rendered into a set of traces 50) is, preferably, vacuum deposited. This plate 50 also may be adhered by way of a 200 Å layer of vacuum deposited chrome (not shown). Plating 50 must be highly conductive and, if vacuum coating is used, must be an element of the periodic table. Accordingly, gold, platinum and iridium are among the materials that may be used. Other deposition techniques, such as chemical deposition, may permit the application of other highly conductive materials, such as a conductive polymer. The material used to create plating 50 must also be susceptible to removal by laser ablating or an etching process.

Next, plate 50 is sectioned into 24 longitudinal traces 50 (other numbers of traces 50 are possible) extending from approximately the tip 20 to the proximal end of base core 14. Accordingly, near the tip 20 the traces 50 have a pitch of about 27 μm, near the base 24 have a pitch of about 80 μm at the proximal end of handle 14 have a pitch of about 630 μm. Of particular utility for performing the task of sectioning the conductive plate into traces 50 is a frequency multiplied ND:YAG laser, which can cut kerfs to separate the traces on the order of 5-10 μm width.

In one preferred embodiment there are just four traces 50. Using this embodiment a compound probing device may be built that incorporates an array of probe assemblies 10 to sense and/or stimulate a number of neural sites separated not just in depth, but also transversely to probe assembly 10 longitudinal dimension.

Next, the conductive traces 50 are coated with an outer layer 60 of high coefficient dielectric material. An additional dip coat of epoxy #377 is one way of accomplishing this. As an alternative, an acrylated epoxy urethane may be used, similar to the acrylated epoxy that may be used for layer 30, and described by Table II, below:

TABLE II

| Substance | Proportion | Source, Contact Information |
|---|---|---|
| Photomer 3015 | 100 PHR | Cognis Corporation, |
| | | http://www.na.cognis.com/northamerica/nacognis.html |
| TMPEOTA, | 50 PHR | Sartomer Company, Inc., |
| (Trimethylolpropane | | http://www.sartomer.com |
| triacrylate SR-351) | | |
| RX 03961 (acrylated | 32 PHR | UCB Radcure, Inc., http://www.chemicals.ucb- |
| urethane) | | group.com/default2.html |
| R-812S (fumed | 10 PHR | Degussa Corp., |
| silica) | | http://www.degussa.com/en/home.html |
| MIBK (Methyl | 63 PHR | Aldrich Co., |
| Isobutyl Keytone) | | http://www.sigmaaldrich.com/Brands/Aldrich.html |
| Darocure 1173 | 2.6 PHR | EM Chemicals, |
| (Photoinitiator) | | http://www.emdchemicals.com/corporate/emd_corporate.asp |

Another method is a vacuum deposition of glass or quartz crystal placed, again over an intermediate 200 Å layer of chrome. Dielectric layer 60 preferably has a thickness of from 10 to 40 um to avoid damage by static electric discharge. A laser is used to ablate this outer layer to create several apertures extending through layer 60, having a diameter of about 10 μm at each prospective microelectrode site. A platinum-iridium electrode or neural contact site 62 is built up, preferably by electroplating, at each of these sites. Other materials that could be used for the neural contact sites 62 are platinum (not mixed with iridium), iridium, and oxidized iridium, which is also referred to as iridium black, and intrinsically conductive polymers, such as a doped polypyrrole.

Base 14 is attached to a plate 70 that includes outwardly extending conductive traces (not shown) that connect traces 50 to a set of connector pins 72. In turn, a set of connectors 72 on plate 70 attach to a matching set of connectors 74 on a circuit card assembly 80. Assembly 80 includes a set of twenty-four circuit cards 82, one for each trace, each bearing an identical amplification circuit for processing each signal from each trace 50 in an identical manner.

The advantages of the above described preferred embodiment should now be apparent. Probe assembly 10 is strong, smooth and sleek, for moving through brain tissue to the site of interest. The cross capacitance between traces 50 is minimized due to the shape of the traces 50, which are curved, solid rectangles, on the order of 0.5 um thick but varying between 10 um and 50 um wide. Finally, identical circuits 82 ensure equal treatment for each trace signal.

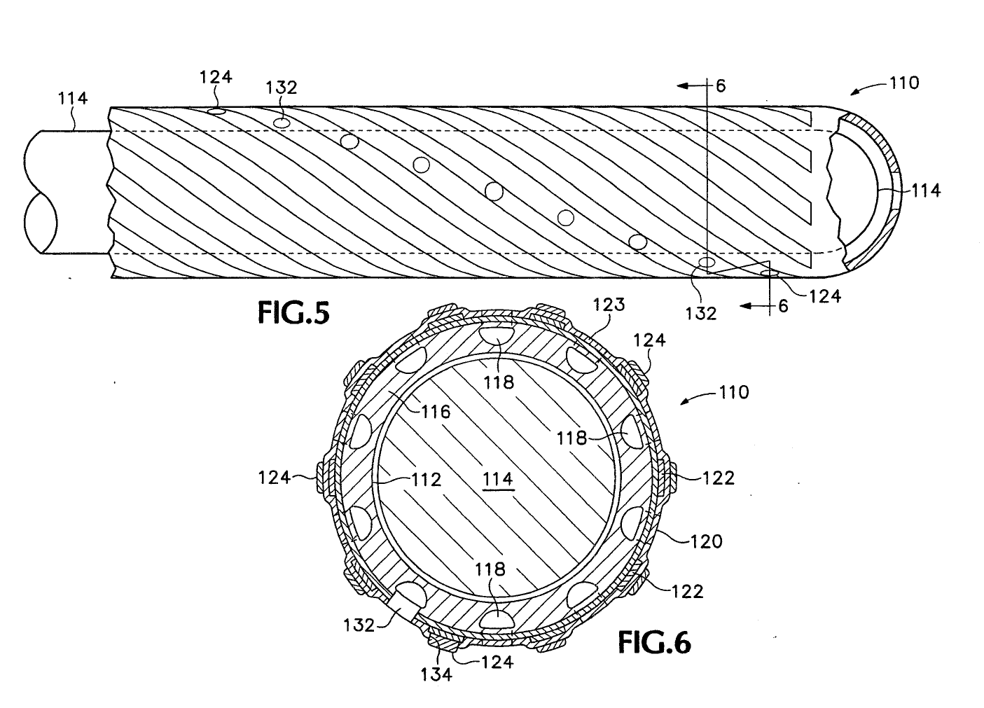

An alternative preferred embodiment of a bio-probe 110 according to the present invention is shown in FIGS. 5 and 6. Bio-probe 110 differs from bio-probe 10 in that it is made of flexible material and defines an inner lumen 112, for accepting a rigid insert 114. Rigid insert 114 permits bio-probe 110 to be pushed through body tissue, for example brain tissue. Insert 114 is then removed, so that as the probe recipient moves about with the probe installed, the flexible bio-probe 110 will not tear into brain tissue, as the brain moves about slightly in the brain pan.

To manufacture bio-probe 110, a mandrel, very similar in nature to insert 114 is used. A tube 116 of flexible dielectric material, for example, polyimide is provided and fit over mandrel 114. Tube 116 defines ten lumens 118, the purpose of lumens 118 will be described later. The production of tube 116 may be effected by molding of polymeric resin. For example tube 116 could be produced by vacuum molding of polyimide resin.

A layer of conductive material, for example gold, is then deposited by, for example, vapor deposition or sputtering. The original deposition of thin layer of conductive material may be followed by an electroplating stage, in which a thicker layer of conductive material is built up on the seed layer.

Next a set of kerfs 120 are created, thereby creating a set of separated conductive traces 122. Kerfs 120 may be formed by laser machining as noted above in reference to bio-probe 10 or through a photolithographic technique. The photolithographic technique could include a mask being pulled across a light source as bio-probe 10, coated with photo resist, is rotated to expose different sections. Other than this rotation technique, the photolithography would be relatively standard, with either positive or negative photo resist being used, and the metal being etched away in places where the developed photo resist has been removed.

Next, an additional layer 123 of dielectric material is coated over traces 122. Apertures 132 are created to lumens 118 and apertures 134 are created to traces 122 by the use of an ND:YAG frequency multiplied laser. Finally, platinum-iridium electrodes 124 are built up in apertures 134. These electrodes are used to stimulate brain cells and sense brain activity. Lumens 118 and apertures 134 are used in the delivery of substances, for example, a medicine or a stimulant to brain tissue. Apertures 132 and electrodes 124 can be used in tandem with a liquid substance administered through apertures 132 and the resultant effect measured by electrodes 124.

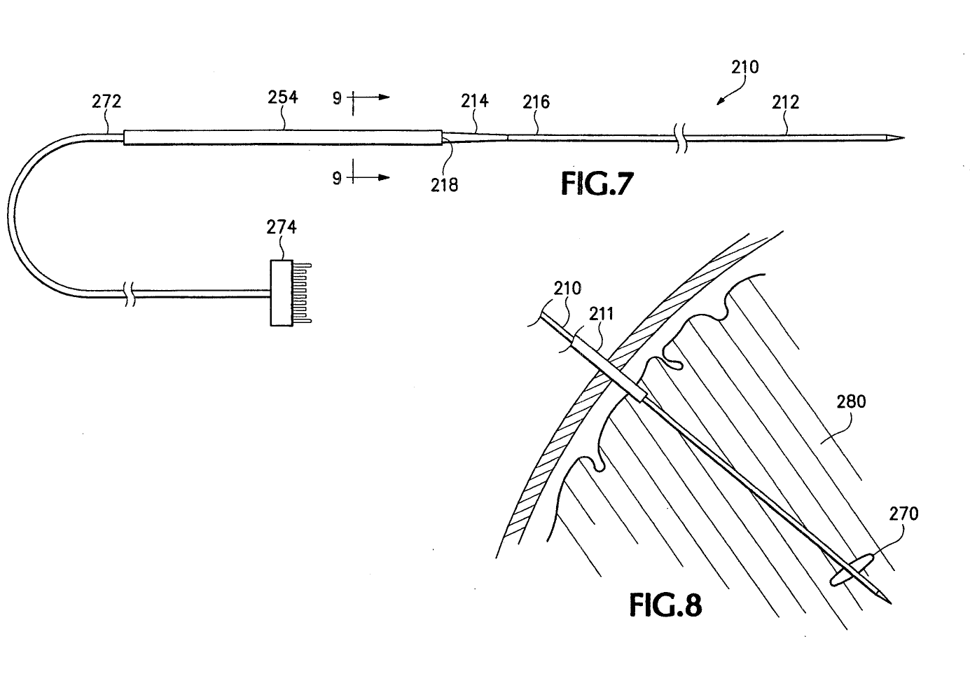

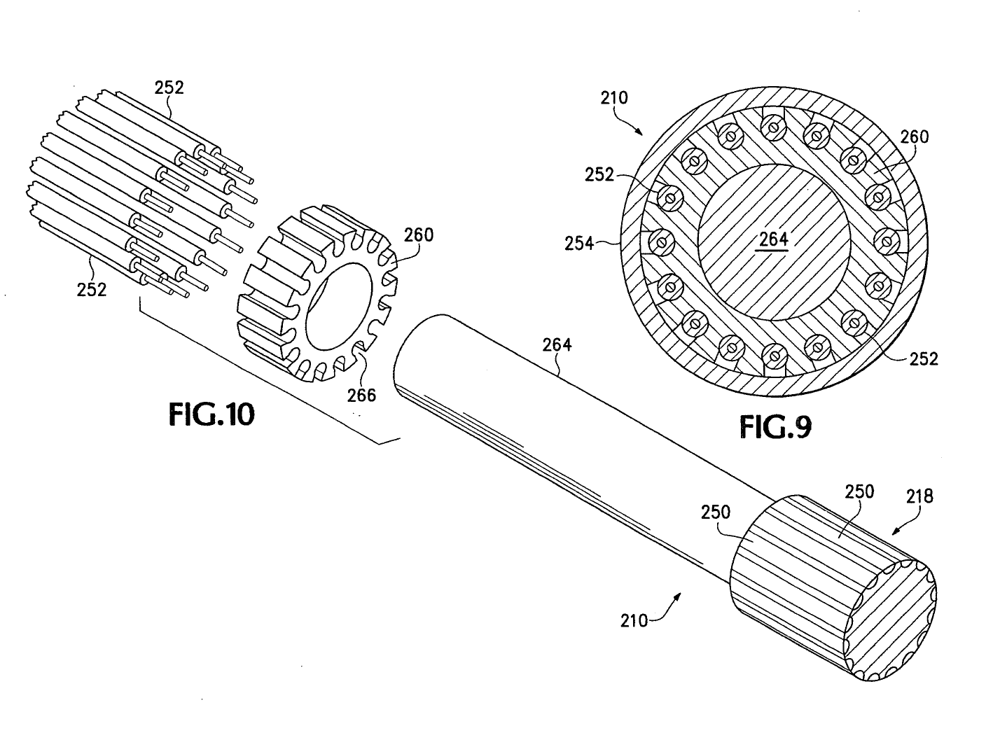

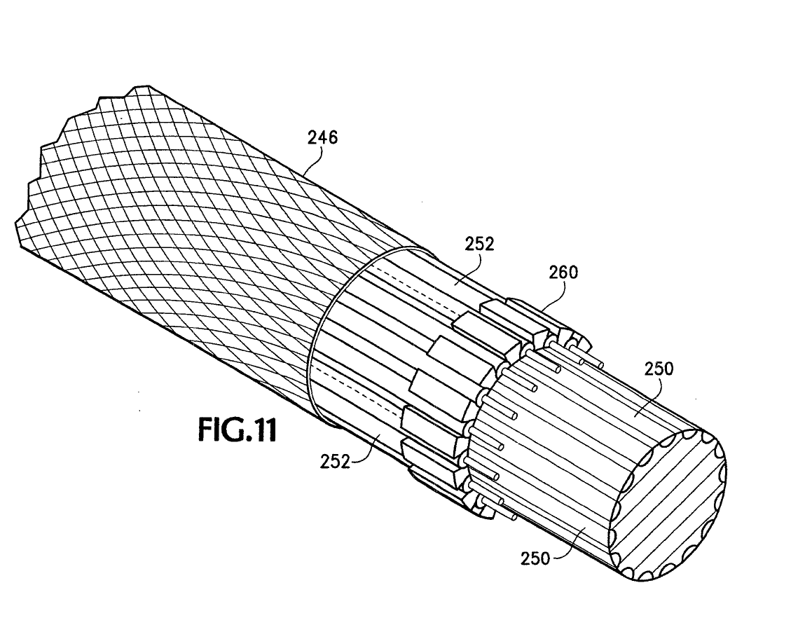

Referring to FIGS. 7-11, an additional preferred embodiment of a brain probe 210 is configured to be thin enough to be inserted into the brain through a canula 211 (FIG. 8). The base 14 of embodiment 10 is eliminated from the design, and each trace 250 (FIGS. 10 and 11) of tip portion 212 (similar in function to tip 12) is terminated to an insulated wire 252. Insulated wires 252 are wrapped together and held in a grounded braided shield 246 (FIG. 11) that is, in turn, contained in a stainless steel tube 254 (FIG. 7) that is grounded by way of the braided shield 246.

The attachment and electrical connection of wires 252 to the traces of tip portion 212 is a challenging operation in which delicate small scale soldering or welding must be performed. Accordingly, it is advantageous to increase the diameter of tip portion 212 as it extends from distal end 216 to proximal end 218. A step-up extent 214 has a changing diameter in the core of the tip portion 212. Having a step-up extent 214 in the core of the tip portion 212 appears to be the most efficient way to accommodate the need for having a very thin distal end 216, for precise placement, and a thicker proximal end 218 for wire termination.

To facilitate the termination of wires 252 to traces 250, a wire holding ring 260 having a set of wire holding keyhole apertures 266 is used. Ring 260 is threaded onto a round hilt 264 of tip portion 212 with insulated wires held in each keyhole 266. This permits the wires 252 to be held in place during the soldering or welding operation, greatly facilitating this operation. After the wire termination is complete, stainless steel tube 254 is slid over wires 252 and, in one preferred embodiment, terminated at ring 260.

Embodiment 210 can be adapted for use with a simian skull and brain, for research activities. Alternatively, a longer variant of preferred embodiment 210 is adapted for use on a human patient and may be used for treating Parkinson's disease, by stimulating the subthalamus nucleus 270 (FIG. 8). Insulated wires 252 form a cable 272 and are connected, by way of connector 274, to a standard amplification unit (not shown), adapted for this purpose.

To use probe 210, an aperture is created in the skull and the canula 211, sized to accept probe 210, is inserted. Then, probe 210 is passed through the canula 211 and into brain tissue 280.

The terms and expressions that have been employed in the foregoing specification are used as terms of description and not of limitation. The term “shield” means an electromagnetic shield. There is no intention, in the use of such terms and expressions, of excluding equivalents of the features shown and described or portions thereof, it being recognized that the scope of the invention is defined and limited only by the claims which follow.

1-20. (canceled)

21. A brain probe for insertion into brain tissue and for sensing neuronal signals, comprising:
(a) a core providing structural support for the brain probe;
(b) a dielectric coating, disposed over the core, a surface of the dielectric coating defining a diameter that tapers from a distal end of the brain probe toward a proximal end of the brain probe, the surface of the dielectric coating being substantially round;
(c) a set of traces circumscribing the dielectric coating, wherein the set of traces are formed by conductive material deposited on or plated to the dielectric coating; and
(d) a dielectric layer applied over the traces, wherein an electrical contact point is exposed through the dielectric layer at a the distal end of the brain probe for each trace of the set of traces.

22. The brain probe of claim 21 wherein the dielectric coating is a flexible tube of insulative material and the core is removable from the tube.

23. The brain probe of claim 21 wherein the set of traces are arranged in a longitudinal manner over the dielectric coating.

24. The brain probe of claim 21 wherein the set of traces are arranged in a helical manner about the dielectric coating.

25. The brain probe of claim 21 further comprising:
(e) a set of insulated wires, each electrically connected to a respective trace of the set of traces; and
(f) a tube affixed to the proximal end of the core, covering the wires.

26. The brain probe of claim 25, wherein the insulated wires have a longitudinal extent and are braided together to form a cable, over at least a portion of their longitudinal extent.

27. The brain probe of claim 26, wherein the cable is covered with a grounded shield.

28. The brain probe of claim 27, wherein the tube is grounded to the grounded shield.

29. The brain probe of claim 25 further comprising:
(g) a ring at a proximal or medial portion of the brain probe, the ring deflning a plurality of openings, wherein each wire of the set of insulated wires traverses through a respective opening of the plurality of openings and is electrically connected to a respective trace of the set of traces at a respective location adjacent to the ring

30. The brain probe of claim 21, wherein each trace of the set of traces has a thickness on the order of 0.5 μm.

31. The brain probe of claim 21, wherein adjacent traces of the set of traces are separated by a distance of approximately 5.0-10.0 μm.

32. The brain probe of claim 21 further comprising a set of plated electrodes with each plated electrode disposed at a respective electrical contact point of the set of traces.

33. The brain probe of claim 21 wherein a diameter of the brain probe at a proximal end of the brain probe is about 2 mm or less.
